(19) United States
(12) Patent Application Publication
(10) Pub. No.: US 2026/0097059 A1
(43) Pub. Date: Apr. 9, 2026

(54) PROCESSES AND COMPOSITIONS WITH ANTIBACTERIAL PROPERTIES FOR THE TREATMENT OF DEMODEX BLEPHARITIS

(71) Applicant: Rolando Toyos, Memphis, TN (US)

(72) Inventor: Rolando Toyos, Memphis, TN (US)

(21) Appl. No.: 19/040,993

(22) Filed: Jan. 30, 2025

Related U.S. Application Data
(60) Provisional application No. 63/702,840, filed on Oct. 3, 2024.

(51) Int. Cl. A61K 31/7048; A61K 31/14; A61K 31/4045; A61P 17/00; A61P 33/14

(52) U.S. Cl. CPC A61K 31/7048; A61K 31/14; A61K 31/4045; A61P 17/00; A61P 33/14

(57) ABSTRACT

Provided herein are compositions for the prevention and treatment of demodex blepharitis in a subject and processes of treatment or prevention of demodex blepharitis. The compositions may include at least one avermectin compound selected from 5-O-demethyl-22,23-dihydroavermectin A₁ₐ or 5-O-demethyl-25-de(1-methylpropyl)-22,23-dihydro-25-(1-methylethyl)avermectin A₁ₐ, or a combination thereof, and melatonin, wherein an aggregate concentration of the avermectin compound is optionally less than 1 wt %. Further devices are provided that include wipe including a woven or non-woven membrane and a treatment composition, the treatment composition including melatonin, one or more anti-microbial compounds, and optionally one or more pharmaceutically acceptable excipients and processes of treatment or prevention of demodex blepharitis in a subject using the wipe, alone or optionally further with the composition as provided herein.

### CROSS-REFERENCE TO RELATED APPLICATIONS

This application depends from and claims priority to U.S. Provisional Application No: 63/702840, filed Oct. 3, 2024, the entire contents of which are incorporated herein by reference.

### TECHNICAL FIELD

The present specification relates generally to treatment or prevention of bacterial infection.

### BACKGROUND

Dry Eye Disease (DED) is increasing throughout the world causing an increase in related diseases like blepharitis. Blepharitis is characterized by inflammation and infection of the eyelids. Usually, dysfunction of the glands that produce the normal tear leads to eyelid ailments. The normal tear provides molecules that are anti-microbial in reducing the bacteria and demodex load and decreasing the incidence of blepharitis. Treatment of DED improves the glands and tears but often blepharitis can persist. Blepharitis is the most difficult problem to treat in the DED patient and, if not controlled, may affect overall gland function. Antibiotic drops, steroids, lid wipes, prescription medications, oral antibiotics, and mechanical treatments have been tried in the past but have failed to eradicate blepharitis in a majority of patients. Accordingly, a further treatment is needed to restore the eyelid to a healthy state.

### SUMMARY

The following summary is provided to facilitate an understanding of some of the innovative features unique to the present disclosure and is not intended to be a full description. A full appreciation of the various aspects of the disclosure can be gained by taking the entire specification, claims, drawings, and abstract as a whole.

The present disclosure concerns processes and compositions for the treatment and/or prevention of demodex blepharitis and/or bacteria around the eye and/or eyelid in a subject. As such, according to some aspects, provided are processes for treatment or prevention of demodex blepharitis including administering to a subject in need thereof a composition including at least one avermectin compound selected from 5-O-dimethyl-22,23-dihydroavermectin A₁ₐ or 5-O-demethyl-25-de(1-methylpropyl)-22,23-dihydro-25-(1-methylethyl)avermectin A₁ₐ, or a combination thereof, and melatonin, wherein an aggregate concentration of the avermectin compound is optionally less than 1 wt %. The composition optionally further includes one or more pharmaceutically acceptable excipients. The melatonin is optionally present at about 0.1 wt % to about 5 wt %. Optionally, the composition includes about 90 mol % 5-O-demethyl-22,23-dihydroavermectin A₁ₐ and about 10 mol % 5-O-demethyl-25-de(1-methylpropyl)-22,23-dihydro-25-(1-methylethyl)avermectin A₁ₐ. Optionally, the aggregate concentration of the avermectin compound is less than about 0.5 wt %, optionally about 0.1 wt %. In the processes as provided herein, the administering is optionally once daily. Optionally, the administering is for 2 hours or more, optionally for 7 hours or more. In some aspects, the administering occurs once daily for 20 or more days. Optionally, the administering includes applying the composition to an upper eyelid, a lower eyelid, or both of the subject.

Also provided are processes for treatment or prevention of demodex blepharitis in a subject including applying a wipe to an upper eyelid, a lower eyelid, or both of the subject, the wipe including a woven or non-woven membrane, and a treatment composition, the treatment composition including melatonin, one or more anti-microbial compounds, and optionally one or more pharmaceutically acceptable excipients. Optionally, the antimicrobial compound includes hydroxypropyl bis-hydroxyethyldimonium chloride. In some aspects, the process further includes applying a second composition including at least one avermectin compound selected from 5-O-demethyl-22,23-dihydroavermectin A₁ₐ, 5-O-demethyl-25-de(1-methylpropyl)-22,23-dihydro-25-(1-methylethyl)avermectin A₁ₐ, or a combination thereof and further including melatonin, wherein an aggregate concentration of the avermectin compound is less than 1 wt %. Optionally, the wipe is applied at a time different than the applying of the composition. Optionally, the wipe is applied in the morning, evening or both.

Also provided are compositions for the treatment of demodex blepharitis in a subject including at least one avermectin compound selected from 5-O-demethyl-22,23-dihydroavermectin A₁ₐ or 5-O-demethyl-25-de(1-methylpropyl)-22,23-dihydro-25-(1-methylethyl)avermectin A₁ₐ, or a combination thereof, and melatonin, wherein an aggregate concentration of the avermectin compound is optionally less than 1 wt %. The composition optionally further includes one or more pharmaceutically acceptable excipients. The melatonin is optionally present at about 0.1 wt % to about 5 wt %. Optionally, the composition includes about 90 mol % 5-O-demethyl-22,23-dihydroavermectin A₁ₐ and about 10 mol % 5-O-demethyl-25-de(1-methylpropyl)-22,23-dihydro-25-(1-methylethyl)avermectin A₁ₐ. Optionally, the aggregate concentration of the avermectin compound is less than about 0.5 wt %, optionally about 0.1 wt %.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description and claims that follow.

### DETAILED DESCRIPTION

The details of embodiments of the presently-disclosed subject matter are set forth in this document. Modifications to embodiments described in this document, and other embodiments, will be evident to those of ordinary skill in the art after a study of the information provided in this document.

Embodiments of the present disclosure generally relate to compositions, devices and processes for the treatment of demodex blepharitis. In some aspects, the compositions, devices and processes set forth herein are beneficial for treating, preventing, and/or addressing demodex blepharitis and/or the presence of demodex mites and bacterial they carry in or around the eye. These and other features and embodiments of the compositions, devices and processes are disclosed in greater detail herein.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about. ” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the”include plural references unless the content clearly dictates otherwise.

As used herein, the term “subject” generally refers to a living being (e.g., animal or human) capable of suffering from demodex blepharitis. In a specific embodiment, the subject is a mammal, such as a human, rat, mouse, monkey, horse, cow, pig, dog, cat, guinea pig, etc. Optionally, the subject is a human subject.

The terms “treat,” “treatment,” and “treating,” as used herein, refer to a method of alleviating or abrogating a disease, disorder, and/or symptoms thereof. Optionally, the disease or disorder is dry eye disease (DED). Optionally, the disease or disorder is demodex blepharitis.

As used herein, the terms “administer” or “administration” may include administration routes such as topical, intraocular, transdermal, or systemic administration, so long as the route of administration results in treatment of dry eye disease and/or demodex blepharitis.

“Effective amount,” as used herein, refers to an amount of an agent sufficient to achieve a desired biological effect. Effective amounts will vary based on a subject's age, body weight, condition, and the like, and may be determined by one of skill in the art in view of the present disclosure. The compositions of the present disclosure can be administered by either single or multiple dosages of an effective amount. In aspects, the effective amount of an agent is an amount sufficient to treat dry eye disease and/or demodex blepharitis. In some aspects, the effective amount is an amount sufficient to prevent, stop, inhibit, or suppress the symptoms of dry eye disease and/or demodex blepharitis.

As described herein, aspects of the present disclosure generally relate to compositions, devices and processes for the treatment of dry eye disease and/or demodex blepharitis in a subject in need thereof, optionally by administering the compositions described in greater detail herein or portions thereof. Optionally, the compositions of the present disclosure include a macrocyclic lactone and melatonin. The compositions described herein may be applied topically to the ocular or periocular area of a subject suffering from demodex blepharitis. Without being bound by theory, in some aspects, the composition is applied without contacting the eye itself.

Demodex is a nocturnal ectoparasite that primarily feeds at night and seeks shelter during the day, making it challenging to target and eliminate. Lacking visual organs, demodex rely on melatonin receptors to detect environmental changes. During the night, melatonin levels naturally rise in human skin, signaling the demodex to emerge from their hiding places to feed. By incorporating melatonin in the composition or devices used herein, the demodex are drawn out from pores within the skin to the surface, rendering them vulnerable and prone to treatment and elimination.

A composition as provided herein may include on or more macrocyclic lactones. Optionally, a macrocyclic lactone is an avermectin compound. Optionally, the avermectin compound is one or more of 5-O-demethyl-22,23-dihydroavermectin A₁ₐ or 5-O-demethyl-25-de(1-methylpropyl)-22,23-dihydro-25-(1-methylethyl) avermectin A₁ₐ. In some aspects, the composition includes at least one of 5-O-demethyl-22,23-dihydroavermectin A₁ₐ or 5-O-demethyl-25-de(1-methylpropyl)-22,23-dihydro-25-(1-methylethyl)avermectin A₁ₐ. Optionally, a composition includes 5-O-demethyl-22,23-dihydroavermectin A₁ₐ and 5-O-demethyl-25-de(1-methylpropyl)-22,23-dihydro-25-(1-methylethyl) avermectin A₁ₐ. In some aspects, the composition of the at least one avermectin and melatonin includes ivermectin and melatonin. In some aspects, the composition of the at least one avermectin and melatonin includes melatonin, 5-O-demethyl-22,23-dihydroavermectin A₁ₐ, and 5-O-demethyl-25-de(1-methylpropyl)-22,23-dihydro-25-(1-methylethyl)avermectin A₁ₐ.

Optionally in some compositions as provided herein or more avermectin compounds are present. The at least one avermectin may include 5-O-demethyl-22,23-dihydroavermectin A₁ₐ and/or 5-O-demethyl-25-de(1-methylpropyl)-22,23-dihydro-25-(1-methylethyl)avermectin A₁ₐ. Optionally, the avermectin compound is as is found in the FDA-approved drug ivermectin. Ivermectin is understood to refer to a combination of two active avermectin compounds: 5-O-demethyl-22,23-dihydroavermectin A₁ₐ and 5-O-demethyl-25-de(1-methylpropyl)-22,23-dihydro-25-(1-methylethyl)avermectin A₁ₐ. Ivermectin is often available as a cream or ointment, such as SOOLANTRA®, and is understood in the art to be effective at a concentration of 1% or higher. Previous attempts at application of ivermectin to address demodex blepharitis have, however, been severely impacted by irritation of the thin eyelid dermis at the effective dose (i.e. 1% or higher). It is an aspect of the present disclosure that when used in combination with melatonin, a lower concentration of the one or more avermectin compounds is effective against demodex blepharitis. The compositions and processes as provided herein may include at least one of 5-O-demethyl-22,23-dihydroavermectin A₁ₐ and 5-O-demethyl-25-de(1-methylpropyl)-22,23-dihydro-25-(1-methylethyl)avermectin A₁ₐ, and melatonin.

The composition of at least one avermectin and melatonin may be a combination of 5-O-demethyl-22,23-dihydroavermectin A₁ₐ and 5-O-demethyl-25-de(1-methylpropyl)-22,23-dihydro-25-(1-methylethyl)avermectin A₁ₐ. Optionally, the 5-O-demethyl-22,23-dihydroavermectin A₁ₐ and 5-O-demethyl-25-de(1-methylpropyl)-22,23-dihydro-25-(1-methylethyl)avermectin A₁ₐ, are present in the composition at a molar ratio of about 9:1 to about 1:9, including about 8:1, 7:1, 6:1, 5:1, 4:1, 3:1, 2:1, 1:1, 1:2, 1:3, 1:4, 1:5, 1:6, 1:7, and 1:8. In some aspects such a ratio can be expressed as mol %. The combination of 5-O-demethyl-22,23-dihydroavermectin A₁ₐ and 5-O-demethyl-25-de(1-methylpropyl)-22,23-dihydro-25-(1-methylethyl)avermectin A₁ₐ may be about 90 mol % 5-O-demethyl-22,23-dihydroavermectin A₁ₐ and about 10 mol % 5-O-demethyl-25-de(1-methylpropyl)-22,23-dihydro-25-(1-methylethyl)avermectin A₁ₐ (ivermectin).

The avermectin compound may be present in the composition at an aggregate concentration (sum of the avermectin compounds in the composition) of less than 1 wt %. Optionally, 5-O-demethyl-22,23-dihydroavermectin A₁ₐ and/or 5-O-demethyl-25-de(1-methylpropyl)-22,23-dihydro-25-(1-methylethyl)avermectin A₁ₐ is present at an aggregate concentration of less than 1 wt %. In some aspects, the aggregate concentration of the avermectin compound is less than about 0.5 wt %. In some aspects, the aggregate concentration of the avermectin compound is about 0.1 wt %. Optionally, the aggregate concentration of avermectin compounds is less than or equal to 0.9 wt %, optionally 0.8 wt %, optionally 0.7 wt %, optionally 0.6 wt %, optionally 0.5 wt %, optionally 0.4 wt %, optionally 0.3 wt %, optionally 0.2 wt %, optionally 0.1 wt %.

A composition as provided herein may optionally further include one or more pharmaceutically acceptable excipients. An excipient may be any component suitable for ocular and/or periocular administration. Suitable excipients may be found in regional pharmacopeia, such as the United States Pharmacopeia, British Pharmacopoeia, British Pharmaceutical Codex, European Pharmacopoeia, Japanese Pharmacopoeia, Korean Pharmacopeia and the like. One or more pharmaceutically acceptable excipients may be present in a composition at a weight percent of greater than zero to 99.9 wt %, or any value or range there between.

Pharmaceutically acceptable excipients are understood in the art to provide no biological or otherwise undesirable effect while serving as a vehicle for delivery of one or more active agents as set forth herein. Such are understood in the art (see, e.g. Handbook of Pharmaceutical Excipients, 9^{th} Ed. 2020, Sheskey et al. eds.; Remington: The Science and Practice of Pharmacy, 23^{rd} Ed. 2020, Adejare ed.). Exemplary excipients may include oils and lipids (e.g., mineral oil, medium chain triglycerides, lanolin, etc.), waxes, silicone-based compounds, hydrophobic polymers, fatty alcohols, emollients and/or stabilizers.

An excipient is optionally a carrier. In some aspects, a carrier is one or more volatile compounds such as volatile silicones. As used herein a “volatile compound” refers to a compound that is capable of evaporating quickly at room temperature (approximately 25° C.). A volatile silicone may illustratively be a cyclic silicone or non-cyclic (linear) silicone. Examples of cyclic silicones illustratively include cyclic polydiorganosiloxanes, cyclotetradimethicones and cyclopentadimethicones. Linear organopolysiloxanes are illustratively alkyl-, alkoxy-or phenyldimethicones, and alkyl-, alkoxy-or phenyltrimethicones. Optionally, a carrier is an aliphatic volatile silicone. Aliphatic volatile silicones optionally have from two to six silicon atoms. Optionally, an aliphatic volatile silicone is a linear polyorganosiloxane such as a polyorganosiloxane with 2 to 6 silicon atoms, optionally, trisiloxane. Optionally, an aliphatic volatile silicone is a cyclic polyorganosiloxane such as a polyorganosiloxane with 2 to 6 silicon atoms, optionally, 2, 3, 4, 5, or 6 silicon atoms. It is appreciated that a composition as provided herein optionally includes more than one carrier.

A composition as provided herein optionally includes as an excipient one or more other additives. One of ordinary skill in the art readily appreciates additives suitable for use with the provided compositions such as to provide desired flow characteristics, absorption, evaporation, delivery of active agent, conversion of a prodrug, or other desired characteristic.

Exemplary other additives include, but are not limited to antioxidants, preservatives, pH buffers, anti-microbial agents, conditioners, stabilizers, vitamins, anti-irritants, chelating agents, surfactants, emollients, thickening agents, humectants, salts, combinations thereof, and the like. Exemplary antioxidants include vitamin E (tocopherol), butylated hydroxytoluene (BHT), ascorbic acid, ascorbyl palmitate, butylated hydroxyanisole, glutathione, ubiquinone, alpha-lipoic acid, green tea extract, citric acid, derivatives thereof, combinations thereof, and the like. Illustrative examples of an anti-irritant include allantonin, derivatives thereof, combinations thereof, and the like. Illustrative preservatives include caprylyl glycol, ethylhexylglycerin, benzalkonium chloride, chlorobutanol, sorbic acid and salts thereof, 1,2-hexanediol, sodium benzoate, parabens, polyquaternium-1, hydroxybenzoates, ethylenediaminetetraacetic acid (EDTA), phenoxyethanol, derivatives thereof, combinations thereof, and the like. A vitamin is illustratively niacinamide, vitamin A, derivatives thereof, combinations thereof, and the like. pH buffers illustratively include phosphate buffered systems, citrate buffer systems (e.g. sodium citrate), borate buffer systems, acetate buffer systems, lactate buffer systems, THAM, glycine buffer systems, sodium bicarbonate, derivatives thereof, combinations thereof, and the like. Exemplary conditioners include but are not limited to hydroxypropyl bis-hydroxyethyldimonium chloride derivatives thereof, combinations thereof, and the like. Exemplary non-limiting surfactants include sodium lauryl sulfate, laureth-4, polysorbates, cremophor, polyoxyl 40 stearate, cetylpyridinium chloride, poloxamers, derivatives thereof, combinations thereof, and the like. In some aspects, the composition includes an emollient, such as petroleum jelly, dimethicone, lanolin, cetearyl alcohol, glycerin, squalane, isopropyl myristate, cocoa butter, shea butter, derivatives thereof, combinations thereof, and the like. Illustratively, a salt is sodium chloride or the like. Illustratively, a humectant is 1,2-hexanediol, saccharide isomerate, sorbitol, derivatives thereof, combinations thereof, and the like. Exemplary non-limiting anti-microbials include hydroxypropyl bis-hydroxyethyldimonium chloride, polyhexamethylene biguanide (PHMB), polyaminopropyl biguanide, chlorohexidine, benzalkonium chloride, sodium perborate, silver sulfadiazine, tobramycin, erythromycin, moxifloxacin, sulfacetaminde sodium, gentamicin, combinations thereof, derivatives thereof, combinations thereof, and the like.

A composition as provided herein optionally includes as an excipient one or more thickeners. A thickener may be one or more dimethicone crosspolymers illustratively dimethicone/vinyl dimethicone crosspolymer and dimethicone/phenyl vinyl dimethicone crosspolymers, high molecular-weight silicone waxes, acrylic polymers that are soluble in silicones, derivatives thereof, combinations thereof, and the like. Illustrative examples of thickeners include polysilicone-11 and stearoxymethicone/dimethicone copolymer.

An excipient as provided herein is optionally a volatile hydrocarbon, such as a volatile high molecular weight hydrocarbon. Illustrative examples of a volatile high molecular weight hydrocarbon vehicle include isododecane, methylpentadecene, and isohexadecane, derivatives thereof, combinations thereof, and the like.

In some aspects, the composition includes a pharmaceutically acceptable neutralizing agent as an excipient. Illustrative examples of neutralizing agents include sodium hydroxide, potassium hydroxide, triethanolamine, ammonium hydroxide, and tris(2-aminoethyl)amine sold by Dow Chemical as TRIS AMINO, derivatives thereof, combinations thereof, and the like.

An excipient is optionally an anti-irritant. Illustrative anti-irritants include licorice and its extracts, dipotassium glycyrrhizinate, tea tree oil, oat and oat extracts, candelilla wax, alpha bisabolol, aloe vera, Manjistha (extracted from plants in the genus Rubia, particularly Rubia cordifolial), and Guggal (extracted from plants in the genus Commiphora, particularly Commiphora Mukul), derivatives thereof, combinations thereof, and the like. It is appreciated that more than one anti-irritant may be included in a composition as provided herein.

In some aspects, the composition may further include a pigment. In some aspects, the pigment may be one or more of titanium dioxide, iron oxides, manganese violet, chromium oxide and ultramarine blue, zinc oxide, D&C Red No. 36 and D&C Orange No. 17, calcium lakes of D&C Red No. 7, 11, 31 and 34, barium lake of D&C Red No. 12, D&C Red No. 13 strontium lake, aluminum lakes of FD&C Yellow No. 5, of FD&C Yellow No. 6, of D&C Red No. 27, of D&C Red No. 21 and FD&C Blue No. 1, derivatives thereof, combinations thereof, and the like.

Optionally, a composition including one or more avermectin compounds and melatonin as provided herein is optionally provided in a pharmaceutically acceptable base. In some aspects, the composition may be formulated as a lotion, cream, gel, bar, or ointment with the pharmaceutically acceptable base. It is appreciated that a lotion may be a water-based formulation that includes emulsifiers to combine water and oil phases, providing a light and easily spreadable texture for topical application. Optionally, a cream may include more emollients to balance hydrophilic and hydrophobic components. A gel formulation may include one or more thickeners to create a composition for administration. The pharmaceutically acceptable base is optionally a PCCA base, optionally PCCA CLARIFYING base (PCCA #30-4845).

A composition as provided herein optionally includes water, optionally is predominantly water (i.e. greater than 50 wt %). Water is optionally present in the composition at 0.1 wt % to 99 wt % or any value or range therebetween. Water is optionally present at about 10 wt %, optionally about 20 wt %, optionally about 30 wt %, optionally about 40 wt %, optionally about 50 wt %, optionally about 60 wt %, optionally about 70 wt %, optionally about 80 wt %, optionally about 90 wt %, or within 1, 2, 3, or 4 wt % above or below any of the foregoing.

In some aspects, melatonin is present in the compositions as set forth herein at about 0.1 wt % to about 5 wt %, including about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, and 4.9 wt %, including any subrange defined by any two of the aforementioned endpoints.

In some aspects, the processes of the present disclosure concern the application or administration of the composition of at least one avermectin compound and melatonin to a subject, optionally to a subject in need thereof who has or is at risk of dry eye disease and/or demodex blepharitis. In some aspects, the composition may be applied and/or administered for the treatment of demodex blepharitis in a subject. In some aspects, the compositions described herein are for topical administration, optionally to the ocular or periocular region of the subject, including the eyelid, eyelash margins, adjacent dermal areas, and/or conjunctiva, to treat conditions affecting these areas. In aspects, the processes of the present disclosure may include application and/or administration of the compositions as set forth herein to an eyelid of a subject. Such administration and/or application may include to an upper eyelid, a lower eyelid, or both of said subject.

In some aspects, the processes of the present disclosure include the administration and/or application of at least one of 5-O-demethyl-22,23-dihydroavermectin A₁ₐ or 5-O-demethyl-25-de(1-methylpropyl)-22,23-dihydro-25-(1-methylethyl)avermectin A₁ₐ, and further includes melatonin. In some aspects, only 5-O-demethyl-22,23-dihydroavermectin A₁ₐ and melatonin are applied. In some aspects, only 5-O-demethyl-25-de(1-methylpropyl)-22,23-dihydro-25-(1-methylethyl)avermectin A₁ₐ and melatonin are applied. In some aspects, 5-O-demethyl-22,23-dihydroavermectin A₁ₐ, 5-O-demethyl-25-de(1-methylpropyl)-22,23-dihydro-25-(1-methylethyl)avermectin A₁ₐ, and melatonin are applied.

It is a further aspect of the present disclosure that the processes herein require less than 1 wt % of aggregate avermectin compounds to yield a desired result when applied with melatonin as described herein. The aggregate concentration of 5-O-demethyl-22,23-dihydroavermectin A₁ₐ and 5-O-demethyl-25-de(1-methylpropyl)-22,23-dihydro-25-(1-methylethyl)avermectin A₁ₐ may be less than 1 wt %.

The at least one avermectin compound and the melatonin may be co-administered. As used herein, “co-administered” refers to administration of the avermectin compound(s) and melatonin, such that administered agents can simultaneously or synergistically achieve a physiological effect, e.g., in a recipient subject. The avermectin compound(s) and melatonin may be administered concurrently, optionally as a single composition. The avermectin compound(s) and melatonin, however, need not be administered together. Administration of one agent can precede administration of the other(s). Simultaneous physiological effect need not necessarily require presence of all agents at the same time. However, in certain embodiments, co-administering typically results in the administered agents being simultaneously present on the application site of the subject. Thus, the avermectin compound(s) and melatonin may be administered concurrently or sequentially.

A period of time may be introduced in between applications of a composition as provided herein, such as a period sufficient to allow absorption of an applied composition prior to application of a further composition. In some aspects, the avermectin compound(s) and the melatonin may be applied at the same time, or separated by a period of time of about one minute to one hundred and twenty minutes.

The administration of the avermectin compound(s) and melatonin allows the composition to remain in contact with the dermis around the subject's eye for a certain duration. Optionally, this duration may range from approximately 10 minutes to about 30 minutes or longer. The duration may last for about two hours or more. In some aspects, administration or application may occur overnight or while the subject is resting. In some aspects, the duration of administration may extend to about 7 hours or more.

The processes of the present disclosure according to some aspects include administration or application of a composition including at least one avermectin compound and melatonin as set forth herein. Optionally, administration or application of at least one avermectin compound and melatonin includes repeated applications. Optionally, administration of the composition is once daily or more, optionally twice, three times, or four times per day, or more. Optionally administration of the avermectin and melatonin is once per day. In some aspects, it may be necessary or desirable to repeat administration or application for a continued period of time, such as daily for about a week or longer, including about two weeks or more, three weeks or more, or for a month or longer. Optionally the avermectin and melatonin is administered once daily for 20 or more days.

In some aspects, the avermectin compound(s) and melatonin may be applied to the ocular or periocular area of a subject for a period of two or more hours and/or may be applied daily.

In further aspects, the compositions and processes as set forth herein may further provide antibacterial properties to an area of application thereof. In some aspects, the compositions and process as set forth herein may provide antibacterial properties when applied to the dermal area surrounding the eye.

In some aspects, the processes described herein include the application of a device that includes one or more agents therein to a subject. Optionally, a device is in the form of a wipe that may be contacted to a target area of a subject. A wipe may include a treatment composition that includes melatonin optionally alone or in combination with one more other agents.

The wipe may be applied the ocular or periocular region of a subject, including the eyelid, eyelash margins, adjacent dermal areas, and/or conjunctiva, to treat conditions affecting these areas. In aspects, the processes of the present disclosure may include application and/or administration of the wipe as set forth herein to an eyelid of a subject. Such administration and/or application may include to an upper eyelid, a lower eyelid, or both of said subject.

Optionally, the wipe includes or is formed from a substrate in the form of a membrane of woven or non-woven material wherein treatment composition may be housed therein or thereon. Optionally, a wipe is formed of a woven material. Optionally, a wipe is formed of a non-woven material. A woven or non-woven material may include formable woven and non-woven synthetic materials or combinations thereof optionally, but not limited to, nylons, synthetic olefins, polyesters, and/or urethanes.

The treatment composition that is present in the device may include melatonin and optionally one or more anti-microbial compounds. The melatonin is optionally present at about 0.1 wt % to about 5 wt %, including about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, and 4.9 wt %, including any subrange defined by any two of the aforementioned endpoints.

Exemplary non-limiting anti-microbials include hydroxypropyl bis-hydroxyethyldimonium chloride, polyhexamethylene biguanide (PHMB), polyaminopropyl biguanide, chlorohexidine, benzalkonium chloride, sodium perborate, silver sulfadiazine, tobramycin, erythromycin, moxifloxacin, sulfacetaminde sodium, gentamicin, combinations thereof, derivatives thereof, combinations thereof, and the like. Optionally, the antimicrobial compound includes hydroxypropyl bis-hydroxyethyldimonium chloride. In some aspects, the wipe further include the composition of at least one avermectin compound and melatonin as otherwise set forth herein.

The treatment composition may also optionally include one or more pharmaceutically acceptable excipients as otherwise provided herein, illustratively but not limited to may include oils and lipids (e.g., mineral oil, medium chain triglycerides, lanolin, etc.), waxes, silicone-based compounds, hydrophobic polymers, fatty alcohols, emollients and/or stabilizers as provided herein.

In some aspects, the processes of the present disclosure may include administration and/or application of the wipe and further administration and/or application of a second composition that is a composition of at least one avermectin compound and melatonin as set forth herein. In some aspects, the wipe is applied at time different than the application of said second composition. In some aspects, the wipe is applied in the morning, the evening or both. Optionally, a second composition is applied in a such a way so as to remain in contact with the dermis around the subject's eye for a certain duration. Optionally, this duration may range from approximately 10 minutes to about 30 minutes or longer. The duration may last for about two hours or more. In some aspects, administration or application may occur overnight or while the subject is resting. In some aspects, the duration of administration may extend to about 7 hours or more.

As indicated above, a process of treating or preventing demodex blepharitis optionally includes application of a wipe as provided herein as well as application of a second composition as provided herein wherein the second composition includes one or more avermectin compounds and melatonin optionally with one or more pharmaceutically acceptable excipients all as otherwise described herein. Optionally, a second composition includes at least one of 5-O-demethyl-22,23-dihydroavermectin A₁ₐ or 5-O-demethyl-25-de(1-methylpropyl)-22,23-dihydro-25-(1-methylethyl)avermectin A₁ₐ, and further includes melatonin. In some aspects, only 5-O-demethyl-22,23-dihydroavermectin A₁ₐ and melatonin are present in a second composition as an active. In some aspects, only 5-O-demethyl-25-de(1-methylpropyl)-22,23-dihydro-25-(1-methylethyl)avermectin A₁ₐ and melatonin are present in a second composition as an active. In some aspects, 5-O-demethyl-22,23-dihydroavermectin A₁ₐ, 5-O-demethyl-25-de(1-methylpropyl)-22,23-dihydro-25-(1-methylethyl)avermectin A₁ₐ, and melatonin as an active. Optionally, the aggregate concentration of 5-O-demethyl-22,23-dihydroavermectin A₁ₐ and 5-O-demethyl-25-de(1-methylpropyl)-22,23-dihydro-25-(1-methylethyl)avermectin A₁ₐ may be less than 1 wt %, optionally less than or equal to 0.9 wt %, optionally 0.8 wt %, optionally 0.7 wt %, optionally 0.6 wt %, optionally 0.5 wt %, optionally 0.4 wt %, optionally 0.3 wt %, optionally 0.2 wt %, optionally 0.1 wt % alone or further in combination with melatonin at about 0.1 wt % to about 5 wt %. Optionally, the second composition includes about 90 mol % 5-O-demethyl-22,23-dihydroavermectin A₁ₐ and about 10 mol % 5-O-demethyl-25-de(1-methylpropyl)-22,23-dihydro-25-(1-methylethyl)avermectin A₁ₐ. Optionally, the aggregate concentration of the avermectin compound is less than about 0.5 wt %, optionally about 0.1 wt %. In the processes as provided herein, the administering of the second composition is optionally once daily. Optionally, the administering of the second composition is for 2 hours or more, optionally for 7 hours or more. In some aspects, the administering occurs once daily for 20 or more days. Optionally, the administering of the second composition includes applying the second composition to an upper eyelid, a lower eyelid, or both of the subject.

It is appreciated that, in addition to causing infection, demodex also contributes to local inflammation. In some aspects, the application of melatonin combined with an antimicrobial offers an additional mechanism to reduce inflammation, while still providing a stimulus to draw demodex to the dermal surface. Without being bound by theory, melatonin may alleviate the inflammation associated with blepharitis and encourage demodex to emerge from hair follicles and pores. The antimicrobial, such as hydroxypropyl bis-hydroxyethyldimonium chloride, serves as an active antimicrobial agent. Optionally, the wipe may be used alongside compositions as described herein (e.g. as a second composition). Alternatively, the wipes may serve as a substitute for other formulations, particularly in cases of severe blepharitis, where the wipes also contain the compositions described herein. In some aspects, the wipes may be used as a maintenance therapy while the subject temporarily pauses or discontinues treatment with the composition containing at least one avermectin compound and melatonin as provided herein.

### EXAMPLES

Several initial formulations of ivermectin were tested in an effort to help DED patients. When ivermectin was used at a concentration of 1 wt % or higher it was found that despite being inserted in a vehicle that is soothing and healing for the skin, the result was that this was insufficient for complete eradication of the demodex and led to unwanted side effects.

It was decided to test the inclusion of melatonin in a composition with the ivermectin as a potential approach to combat the inefficacies of ivermectin alone. Demodex is a nocturnal species, feeding at night and hiding during the day, making it difficult to kill. Demodex have no vision but do possess melatonin receptors. At night melatonin concentrations increase in the skin. The demodex receptors pick up on that increase or melatonin and come out to feed. Thus several formulations of ivermectin and melatonin were tested. The formulation “Ivermectin Plus” (ivermectin 1 wt % and melatonin 1 wt %) has melatonin in the formulation thereby simulating what happens to at night drawing out the demodex for more effective elimination. A second formulation for the eyelids “Ivermectin Plus Lids” (0.1 wt % ivermectin and 1 wt % melatonin) was developed to be used specifically on the eyelids of the patient. Patients can apply Ivermectin Plus (1.0% Ivermectin and 1.0% melatonin) everywhere except the eyelids while Ivermectin Plus Lids is used on the upper and lower eyelids.

Patients apply the cream Ivermectin Plus Lids at night and thoroughly wash off remaining composition in the morning. Since the demodex lifecycle is a two weeks and the mites carry several eggs, the medication can be used for 30 nights to eradicate the problem. After 30 nights the patients can use the medication weekly to keep demodex under control. The formulation(s) can be further applied as anti-inflammatories. Blepharitis is not only an infection but also an inflammation. This treatment eliminates the root cause of the problem and the inflammation the infection causes.

A further wipe was formulated to further assist with inflammation when used alone or in combination with the ivermectin plus lids formulation above. The wipe is an anti-microbial wipe that is effective in treating blepharitis by controlling bacterial and demodex proliferation. The wipe contains melatonin and hydroxypropyl bis-hydroxyethyldimonium chloride. The melatonin decreases the inflammation associated with blepharitis and encourages the demodex to come out of the hair follicles and pores. The hydroxypropyl bis-hydroxyethyldimonium chloride is an active anti-microbial compound. The patients can use the wipe morning and/or night on the eyelids. They can use it instead of Ivermectin Plus, Ivermectin Plus Lids, or alone. Also, in those patients who have used Ivermectin Plus Lids for 30 nights, and now are on maintenance therapy, they can use the wipes as a daily maintenance or preventative therapy.

A retrospective chart review of over 100 patients that have used Ivermectin Plus Lids alone or with the wipes as set forth above revealed a decrease in the signs of blepharitis. In chart reviews of over 30 patients that have used just the Ivermectin Plus Lids and over 30 patients who have only used the wipe, a clinically significant decrease was found in the signs of blepharitis. In a prospective study of 10 patients using Ivermectin Plus Lids and the wipes, a clinically significant decrease was found in the signs of blepharitis. Signs studied included scurf, collarettes, and pouting of the base of the hair follicle. Fewer demodex were also found in patients whose hair follicles were pulled and examined under the microscope.

It is also to be understood that this disclosure is not limited to the specific aspects and methods described herein. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or. ” As used herein, the term “and/or” includes any and all combinations of one or more of the associated listed items.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.” It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A process for treatment or prevention of demodex blepharitis comprising:
administering to a subject in need thereof a composition comprising:
at least one avermectin compound selected from 5-O-demethyl-22,23-dihydroavermectin A₁ₐ, 5-O-demethyl-25-de(1-methylpropyl)-22,23-dihydro-25-(1-methylethyl)avermectin A₁ₐ, or a combination thereof, and
melatonin,
wherein an aggregate concentration of the avermectin compound is less than 1 wt %.

2. The process of claim 1, wherein the composition further comprises one or more pharmaceutically acceptable excipients.

3. The process of claim 1, wherein said melatonin is present at about 0.1 wt % to about 5 wt %.

4. The process of claim 1, wherein the avermectin compound comprises a combination of 5-O-demethyl-22,23-dihydroavermectin A₁ₐ and 5-O-demethyl-25-de(1-methylpropyl)-22,23-dihydro-25-(1-methylethyl)avermectin A₁ₐ.

5. The process of claim 4, wherein the combination is about 90 mol % 5-O-demethyl-22,23-dihydroavermectin A₁ₐ and about 10 mol % 5-O-demethyl-25-de(1-methylpropyl)-22,23-dihydro-25-(1-methylethyl)avermectin A₁ₐ.

6. The process of claim 1, wherein the aggregate concentration is less than about 0.5 wt %.

7. The process of claim 1, wherein the aggregate concentration is about 0.1 wt %.

8. The process of claim 1, wherein said administering occurs once daily.

9. The process of claim 1, wherein said administering is for 2 hours or more.

10. The process of claim 1, wherein said administering is for 7 hours or more.

11. The process of claim 1, wherein said administering occurs once daily for 20 or more days.

12. The process of claim 1, wherein said administering comprises applying the composition to an upper eyelid, a lower eyelid, or both of said subject.

13. A process for treatment or prevention of demodex blepharitis in a subject in need thereof comprising:
applying a wipe to an upper eyelid, a lower eyelid, or both of said subject, said wipe comprising:
a woven or non-woven membrane; and
a treatment composition, the treatment composition comprising melatonin, one or more anti-microbial compounds, and optionally one or more pharmaceutically acceptable excipients.

14. The process of claim 13, wherein said antimicrobial compound comprises hydroxypropyl bis-hydroxyethyldimonium chloride.

15. The process of claim 13, wherein said process further comprises applying a second composition comprising at least one avermectin compound selected from 5-O-demethyl-22,23-dihydroavermectin A₁ₐ or 5-O-demethyl-25-de(1-methylpropyl)-22,23-dihydro-25-(1-methylethyl)avermectin A₁ₐ, or a combination thereof and melatonin, wherein an aggregate concentration of the avermectin compound is less than 1 wt %.

16. The process of claim 15, wherein said wipe is applied at time different than the application of said composition.

17. The process of claim 13, wherein said wipe is applied in the morning, the evening or both.

18. A composition for the treatment of demodex blepharitis in a subject comprising:
at least one avermectin compound selected from of 5-O-demethyl-22,23-dihydroavermectin A₁ₐ and 5-O-demethyl-25-de(1-methylpropyl)-22,23-dihydro-25-(1-methylethyl)avermectin A₁ₐ, and melatonin,
wherein an aggregate concentration of the avermectin compound is less than 1 wt %, and optionally further comprising one or more pharmaceutically acceptable excipients.

19. The composition of claim 18, wherein said melatonin is present at about 0.1 wt % to about 5 wt % 20. The composition of claim 18, wherein the avermectin compound comprises a combination of 5-O-demethyl-22,23-dihydroavermectin A₁ₐ and 5-O-demethyl-25-de(1-methylpropyl)-22,23-dihydro-25-(1-methylethyl)avermectin A₁ₐ, wherein the combination is about 90 mol % 5-O-demethyl-22,23-dihydroavermectin A₁ₐ and about 10 mol % 5-O-demethyl-25-de(1-methylpropyl)-22,23-dihydro-25-(1-methylethyl)avermectin A₁ₐ.
